(19) United States
(12) Patent Application Publication
(10) Pub. No.: US 2025/0249168 A1
(43) Pub. Date: Aug. 7, 2025

(54) MEDICAL INFUSION SYSTEM AND ASSOCIATED OPERATING PROCEDURE

(71) Applicant: B. Braun Melsungen AG, Melsungen (DE)

(72) Inventor: Jan Schwarz, Melsungen (DE)

(21) Appl. No.: 19/040,982

(22) Filed: Jan. 30, 2025

(30) Foreign Application Priority Data
Feb. 1, 2024 (EP) 24155333.8
Jun. 11, 2024 (EP) 24181418.5

(51) Int. Cl. A61M 5/142; A61M 5/172

(52) U.S. Cl. CPC A61M 5/142; A61M 5/172; A61M 2205/273

(57) ABSTRACT

A medical infusion system includes a disposable article and an infusion pump. The disposable article can be inserted into or connected to the infusion pump. The disposable article can be an infusion tube or a syringe. The infusion pump can be a peristaltic pump or a syringe pump. The disposable article contains a machine-readable code or is connected to an accessory carrying a machine-readable code. The machine-readable code contains or represents an expiry period, in particular a retention period, a shelf life, or an expiry date, of the disposable article. The infusion pump has a sensor for detecting the machine-readable code. A pump controller determines the expiry time of the disposable article based on the machine-readable code.

### CROSS-REFERENCE TO RELATED APPLICATIONS

This application claims priority under 35 U.S.C. § 119 to European Application No. 24181418.5, filed on Jun. 11, 2024, and to European Application No. 24155333.8, filed on Feb. 1, 2024. The contents of European Application No. 24181418.5 and European Application No. 24155333.8 are incorporated by reference herein in their entireties.

### FIELD

The invention relates to a medical infusion system with at least one disposable article, in particular an infusion tube or a syringe, provided for insertion into or connection to an infusion device, in particular an infusion pump or a syringe, and with an associated infusion device.

The invention also relates to an operating method for an infusion pump.

### BACKGROUND

The use of disposable items such as infusion tubes in the medical environment is common practice. However, monitoring and ensuring that the shelf life or expiry date of such items is not exceeded is crucial to minimize potential risks to the patient.

For example, an infusion tube for a medical infusion system has a shelf life of a certain time after sterilization, usually 5 years. At the same time, the period of use, and therefore the length of time it remains in an infusion device, is usually limited. For example, the use of an infusion tube in an infusion pump, especially in a peristaltic chewing area, can lead to fatigue of the tube material over time and possibly to a risk of sterility or to a deviating delivery accuracy. Depending on the solutions introduced into the disposable article, it may also be useful to determine a maximum expiry time or dwell time after the infusion device has been put into operation and the disposable article has been inserted, in order to ensure that a disposable article can only be used in the infusion device for such a predetermined time. After the expiry time, in particular the retention time, the shelf life or an expiry date, the infusion tube may no longer be used. However, this requirement is sometimes—intentionally or unintentionally—disregarded.

### SUMMARY

The present invention aims to solve this problem by providing an infusion system and an associated method which enables the expiry time, in particular the dwell time, shelf life or expiry date of disposables to be monitored during system operation. On the one hand, the invention seeks to ensure that only disposables with a valid, unexpired shelf life or expiration date are used, thereby improving patient safety and the effectiveness of medical treatment. On the other hand, the aim of the invention is to ensure that disposable articles are only used until the maximum retention time has been reached, which also improves patient safety and the effectiveness of medical treatment. It should be understood that in the context of the invention, the shelf life or an expiry date on the one hand and/or a maximum retention time on the other hand can each be recorded individually, optionally independently of one another or simultaneously.

Accordingly, a medical infusion system is planned with
at least one disposable article intended for insertion into or connection to an infusion device, in particular a peristaltic pump or a syringe pump, in particular an infusion tube or a syringe, which is provided either itself or by an accessory connected to it with a machine-readable code which contains or represents an expiry time, in particular a retention time or a shelf life or an expiry date, of the disposable article,
at least one infusion device with a receptacle or a connection for the disposable article, furthermore with a sensor for detecting the code, and furthermore with a pump controller which is designed or programmed in such a way that it determines the expiry time, in particular the dwell time, the shelf life or the expiry date, of the disposable article on the basis of the detected code.

The term “expiry period” as used herein preferably refers to a period of time, in particular a predetermined period of time, at the end of which a disposable article should no longer be used.

In particular, it is included that the term “expiry period” in a first alternative preferably means the shelf life or an expiry date. In this context, the term “expiry period” preferably refers to the entire duration from the manufacture of the disposable article until the point in time at which the disposable article should no longer be used, irrespective of its storage or use. In other words, the term “expiry date” in the first alternative preferably refers to a minimum expiration date of the single-use infusion article. This can particularly preferably be seen with colored terminals, similar to a type of quality seal or TÜV seal.

Additionally or alternatively, the term “expiration time” as used herein in a second alternative preferably means a dwell time, i.e. a maximum period of time that the disposable article may be used within the infusion device, possibly with interruptions. According to this second alternative, a dwell time is preferably a maximum total period of use during ongoing delivery/therapy. The retention time according to the second alternative is usually significantly shorter compared to the shelf life according to the first alternative.

It should be understood that in the context of the invention, the shelf life or an expiry date in the sense of a maximum total useful life on the one hand and/or a maximum retention period on the other hand can be recorded individually, optionally independently of each other or simultaneously.

In both cases, the first and the second alternative, the expiry time can preferably be predetermined depending on the material of the disposable article and/or depending on the solutions contained in the disposable article, and possibly also depending on the intended use. Usually, the disposable article, e.g. a syringe or a tube, is initially unfilled and is only filled with a liquid, e.g. a syringe, or connected to an infusion bag, e.g. in the case of a tube, shortly before use. However, the invention is not limited to this and can additionally or alternatively also comprise prefilled articles, whereby an expiry time can then also refer to the dwell time and/or the shelf life of the filling.

In a preferred embodiment of the medical infusion system according to the invention, the pump controller is designed or programmed in such a way that it determines the expiry time, in particular the dwell time, the shelf life or the expiry date, of the disposable article on the basis of the detected code and compares it with a system time and, depending on the result of the comparison, generates a signal to the user which is preferably suitable for blocking or permitting infusion operation with the disposable article and, preferably, for blocking or permitting infusion operation with the disposable article, of the disposable article and compares it with a system time and, depending on the result of the comparison, generates a signal to the user which is preferably suitable for blocking or permitting an infusion operation with the disposable article and, preferably in the event of a blockage, further preferably outputs the signal as a warning message via a user interface.

Advantageously, the pump controller is designed or programmed in such a way that it issues a warning message, such as an acoustic signal and/or an indication on a display, via a user interface in the event of a blockage

In a preferred field of application, the disposable article is an infusion tube, and the accessory is a sliding clamp or other tube clamp regulating the flow of infusion solution through the infusion tube, in particular an “anti-free-flow clamp”, which is actuated by a closure flap covering the receptacle or a similar mechanism when inserted into the infusion pump. In particular, for safety reasons, it may be considered that a clamp is inserted manually and pulled out when the front flap is opened by means of a mechanism, e.g. a hook, in order to prevent free flow when the front flap is opened.

In a preferred embodiment, the code is in the form of a color code on the disposable article or the accessory, and the associated sensor is a color sensor. Advantageously, for the purpose of evaluation, an assignment table is stored in the infusion system, in particular in the pump controller, which links the various colors of the color coding with a respective expiry period, in particular a dwell time, a shelf life or an expiry date. In an advantageous embodiment, such an assignment table can additionally or alternatively also be stored outside the pump controller in or even outside the infusion device. For example, an assignment table can be stored in a cloud and/or on external storage media or servers. Preferably, the assignment table is digitally signed and/or stored in encrypted form.

Alternatively, the code can be a graphic code, in particular a 2D code or a 3D code, or it can be realized by an RFID tag or by forming (mechanical coding). The sensor for detecting or reading the code is then of course adapted to the type of code.

By designing or programming the pump controller to synchronize the system time with an external time, correct and accurate monitoring of the shelf life or expiration date of the disposable is ensured, even in environments where the infusion pump's internal system time is potentially error-prone or not accurate enough.

The features, variants, tasks and advantages mentioned for the device apply analogously to the method and vice versa.

In summary, the invention relates to a concept for coding the expiry time, in particular the dwell time, the shelf life or the expiry date of a disposable article, in particular in the form of an infusion tube or a syringe, in conjunction with an infusion pump or syringe pump, wherein the coding and the infusion device together ensure that the disposable article or the infusion tube or the syringe can no longer be used after the expiry time, in particular the dwell time, the shelf life or the expiry date.

The coding of the disposable article can be carried out in particular by a colored sliding clip or a colored marking on the sliding clip, which is assigned to the disposable article and preferably connected to it.

Alternatively, for example, the disposable article can be coded using a colored component, e.g. a syringe plunger, or a colored marking on the syringe plunger, which is assigned to the disposable article and preferably connected to it.

The color is detected by the infusion device, the infusion pump or the syringe pump by an integrated sensor. A color table is stored within the infusion device, preferably signed and thus protected against changes, which defines an expiry time, in particular a retention time, a shelf life or an expiry date, depending on the color, in each case plus a grace period (for tolerated exceeding of the expiry time), in which the infusion tube can be properly inserted and used within the infusion pump. If the expiration date or the expiry period is exceeded, the infusion pump ensures, preferably by displaying to the user, that the infusion tube can no longer be used because the expiry period, i.e. the retention time, expiry date or use-by date, has been exceeded.

In addition, this concept offers the possibility of protecting the infusion tubing against counterfeiting, because its compatibility with the infusion pump is coded on the one hand by the expiry date and on the other hand by the colored sliding clamp. If the color coding by the manufacturer of the infusion tube is unknown to outsiders, this results in copy protection.

To implement the concept in practice, an infusion pump is equipped with a sensor and an algorithm implemented in suitable software and/or hardware to detect a colored sliding clamp. A colored marking area on the sliding clamp is sufficient for this; the entire sliding clamp does not have to be colored. The infusion pump is therefore able to recognize a different number of colored sliding clamps. The colored slide clamps are thus used to code an expiry period, in particular expiry dates of the infusion tube; similar to a TÜV sticker for the general inspection of motor vehicles. For example, a green clamp can be used in the years 2023 to 2028; a red one in the years 2024 to 2029 etc.

The expiry date can be defined separately depending on the color of the sliding clamp, whereby this definition is conveniently stored in the form of a lookup or mapping table within the infusion pump (i.e. the expiry date can be defined differently depending on the color, possibly with a grace period). If the infusion tube is inserted into the infusion pump, the color of the sliding clamp is recognized by the infusion pump. The infusion pump uses the parameters “detected color” (marking of the slide clamp), the mapping table and the current date known to the infusion pump to assess whether the inserted infusion tube can be used or whether its expiration date has been exceeded or the expiry date has been reached. In the first case (expiry date not reached, i.e. tube usable), the infusion tube can be used by the infusion pump as usual; in the second case (expiry date reached or exceeded), the infusion pump rejects the inserted infusion tube and blocks the use of this single-use device, thereby increasing safety for the patient.

In a similar way, colored sliding clamps can be used to code an expiration time in the form of a dwell time of the infusion tube; similar to a parking disc for motor vehicles. For example, a yellow clamp can indicate a total usage time of 96 hours, an orange clamp can indicate a total usage time of 5 hours, etc.

The dwell time can be defined separately depending on the color of the sliding clamp, whereby this definition is conveniently stored in the form of a lookup or mapping table within the infusion pump (i.e. the dwell time can be defined differently depending on the color, possibly with a grace period). If the infusion tube is inserted into the infusion pump, the color of the sliding clamp is recognized by the infusion pump. The infusion pump uses the parameters “detected color” (marking of the sliding clamp), the mapping table and the current date, which is known to the infusion pump, to assess whether the inserted infusion tube can be used or whether its total dwell time has been exceeded. It is preferred that the pump or a suitable calculation medium, e.g. the control unit, stores a time that has already elapsed and subtracts it from the total dwell time so that a resulting remaining dwell time is determined and the use of the system is either permitted or blocked. A signal can preferably be output to the user, in particular the display of the remaining dwell time as a signal to the user. In the first case (total dwell time not reached, i.e. tube usable), the infusion tube can be used as usual by the infusion pump; in the second case (total dwell time reached or exceeded), the infusion pump rejects the inserted infusion tube and blocks the use of this disposable item, thereby increasing safety for the patient.

The lookup or mapping table is advantageously stored in the infusion pump in a signed, i.e. encrypted, form. This ensures that it is protected against changes and that changes can only be made by authorized personnel of the manufacturer.

Furthermore, the colored sliding clamp can be used in conjunction with the infusion tubing and the infusion pump as copy protection. This concept offers the possibility of the infusion tubing from being copied or imitated because its compatibility with the infusion pump is coded by the expiry date on the one hand and by the colored slide clamp on the other. If the specification of the color coding (i.e. the mapping table) by the manufacturer of the infusion tube is unknown to outsiders, this results in copy protection.

### BRIEF DESCRIPTION OF THE DRAWINGS

Various embodiments of the invention are explained in more detail below with reference to the accompanying drawings.

FIG. 1 is a purely schematic representation of an infusion pump with an infusion tube inserted;

FIG. 2 shows a top view of a sliding clamp for an infusion tube;

FIG. 3 shows, in more detail, an infusion system with an infusion pump designed as a peristaltic pump (peristaltic pump) into which an infusion tube is inserted; and

FIG. 4 shows an infusion system with a syringe pump.

Identical or similar elements are marked with the same reference signs in all figures.

### DETAILED DESCRIPTION

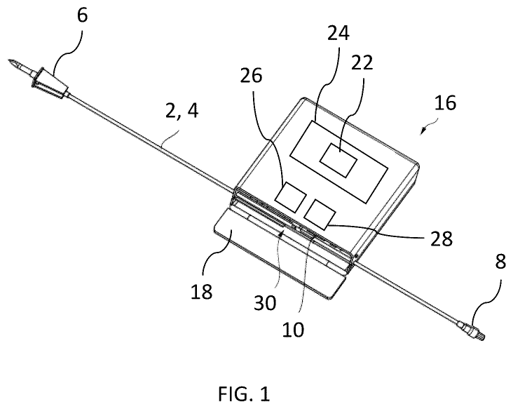

An infusion tube 2, as shown schematically in FIG. 1 as an example of a disposable article 4 within an infusion system, is a medical device used to introduce fluids such as medications, electrolyte solutions or nutrients directly into a patient's body. It plays an important role in intravenous therapy by establishing a connection between the infusion source, in particular an infusion bag with infusion solution, and the patient. In combination with an infusion pump 16, an infusion tube 2 enables precise dosing and administration of substances in order to improve or maintain the patient's state of health.

An infusion tube 2 has two ends that are designed for different purposes:

The drip chamber or infusion needle end 6: A drip chamber is usually connected here, in which the infusion solution drips off and continues to flow into the patient's body. This end of the infusion tube 2 is therefore usually connected via a drip chamber to an infusion needle or an infusion catheter, which is inserted directly into a vein and thus into the patient's bloodstream.

The infusion bag or infusion pump end 8: This end of the infusion tube 2 is connected to an infusion bag or infusion pump 16. The infusion bag or infusion pump 16 contains or supplies the infusion solution and regulates the flow through the infusion tube 2 into the drip chamber.

In summary, the two ends of an infusion tube fulfill 2 different functions: One end is connected to the infusion source, while the other end provides direct access to the patient to deliver the infusion fluid into the body.

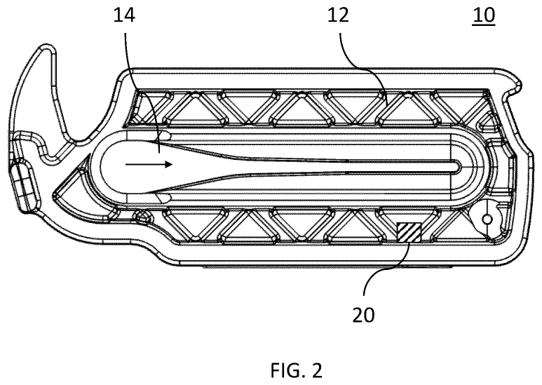

A sliding clamp 10 shown in FIG. 2 is an important accessory in connection with an infusion tube 2. The sliding clamp 10 is normally made of plastic and is pushed onto the infusion tube 2 in order to squeeze it as required and thereby prevent or interrupt the flow of the liquid.

A sliding clamp 10 is shown in FIG. 2 as an example in a detailed top view. According to the above, it serves to pinch the tubing and prevents the free flow of the infusion solution. The sliding clamp 10 has a flat base body or frame 12, typically made of plastic, which encloses an elongated hole 14 that widens from right to left. During operation, the sliding clamp 10 is pushed onto the infusion tube 2 and encloses it so that the infusion tube 2 passes through the elongated hole 14. If the infusion tube 2 is positioned in the wide end of the elongated hole 14, it is not squeezed and has its maximum cross-section (opening position). If, on the other hand, the infusion tube 2 is moved relative to the sliding clamp 10 in the direction of the arrow towards the narrow end of the elongated hole 14, it is increasingly squeezed and finally completely closed (closed position).

An infusion tube 2 of the type mentioned can be designed in particular for insertion into an infusion pump 16 designed as a peristaltic pump. In a peristaltic pump, the infusion tube 2 is periodically compressed and relaxed by rotating rollers or rollers or also by plungers attached to a shaft in order to deliver the liquid. The advantage of such a pump is that the liquid remains in the tube and does not come into contact with the pump mechanism, which prevents contamination of the liquid.

A sliding clamp 10 pushed onto the infusion tube 2 or short tube can be designed as a so-called “anti-free-flow clamp” or “free-flow protection clamp”. This is a safety clamp or clamp for short, which is intended to prevent a free flow of the fluid to be pumped when it is removed from the device (i.e. the infusion pump 16). When the tubing set is delivered, the clamp is open. This is because it must be open during storage, as prolonged storage can cause the tubing to deform or the plastic disposable to deform plastically. The clamp is therefore initially open when it is inserted into the device. When the disposable is removed from the device, the flap pulls the clamp closed again using an eyelet that is hooked into a hook on the clamp and closes the tube to prevent free flow. This is why the clamp is also called a free-flow protection clamp.

For this purpose, the infusion pump 16 can have a suitably shaped receptacle 30 (concealed by the closure flap 30 in FIG. 1) for the infusion tube 2 and the sliding clamp 10, which can be closed by a closure flap 18, whereby the sliding clamp 10 is actuated by opening and closing the closure flap 18 (alternatively by inserting or removing the infusion tube 2 with the sliding clamp 10), as described above. The eyelet for pulling out the sliding clamp 10 is preferably attached in the front flap (closure flap) of the infusion pump 16, which is not visible in the purely schematic FIG. 1, however.

The infusion pump 16 also usually has a clamp on the pump side, which can squeeze the tube when it is inserted and thus also prevent free flow.

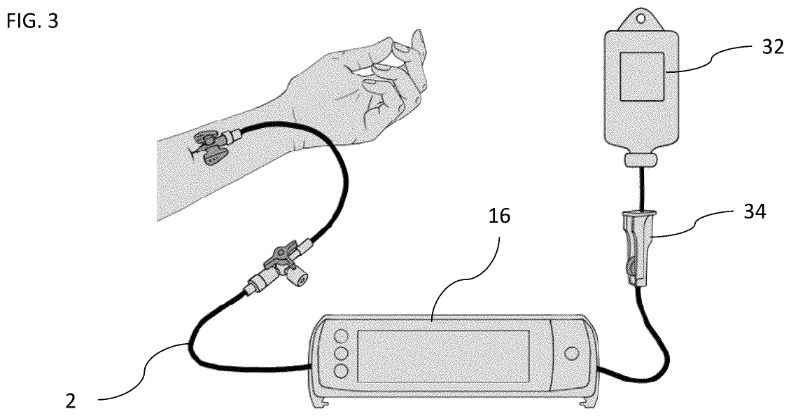

With a sliding clamp 10, it is possible to connect the infusion tube 2 outside the infusion pump 6 to an infusion bag 32, whereby the sliding clamp 10 in the closed position prevents the flow of infusion solution. However, this is only a side effect. The actual closing is usually carried out by a roller clamp 34, as shown in FIG. 3. An overview of the infusion system with infusion bag 32, infusion tube 2, roller clamp 34 and infusion pump 16 is shown there, whereby the infusion tube 2 with the sliding clamp 10 is inserted into the infusion pump 16 (the sliding clamp 10 is not visible here due to the closed closure flap).

For commissioning, the infusion tube 2 is inserted into the infusion pump 16 in a section provided for this purpose together with the sliding clamp 10 with the closure flap 18 open and the closure flap 18 closed. The sliding clamp 10 moves into the open position (whereby the infusion pump 10 now controls the flow of liquid through the infusion tube 2). In other words, the user inserts the clamp into a receptacle with a clamp module, whereby it is/will be opened. In some cases, this is also achieved by closing the front flap, as the mechanical pressure displaces the clamp.

The infusion tube 2 with the sliding clamp 10 already attached is normally stored as a pre-assembled unit in a sterilized state in sterile or hermetically sealed packaging and kept ready for use. After expiry of a predetermined expiry period, in particular the retention time, the shelf life or the expiry date (calculated, for example, from the production or packaging date), possibly taking into account a tolerated excess or grace period, or after exceeding a predetermined expiry date, the infusion tube 2 should no longer be used because then, for example, the sterility of the packaged goods and/or desired material properties can no longer be guaranteed. At the same time, it is also necessary after commissioning and insertion of the disposable article to limit the expiry date with regard to the entire duration of use, i.e. the time that the article is in use in the infusion device, possibly with interruptions, as risk-free use can no longer be guaranteed due to, for example, the solutions contained in the disposable article, material fatigue, e.g. due to peristaltic pumping, or other circumstances. Unfortunately, this requirement is sometimes ignored or unintentionally disregarded in practice.

In order to prevent such undesired use of an infusion tube 2 after expiry of the expiry period, in particular the dwell time, the shelf life or the expiry date, in a preferred embodiment, the sliding clamp 10 connected or connectable to the infusion tube 2 and designed as an “anti-free-flow clamp” in the sense described above for interaction with an associated infusion pump 16 is provided/equipped/labeled or designed at a predetermined location with a machine-readable code which contains or represents the shelf life or the expiry date.

A code is generally a systematic arrangement of characters, symbols, colors, signals or other information carriers that are used to store, transmit, encrypt, decrypt or conceal information. A code enables the storage and transmission of information in a structured and/or coded (especially encrypted) form.

In a preferred embodiment, the code is a color code 20, for example in the form of a (particularly monochrome) color marking or a (particularly multicolored) color pattern at a predetermined location on the frame 12 of the sliding clamp 10. The color used for coding and marking the sliding clamp 10 can be produced, for example, as a color imprint, as a coating, by applying a color element or by suitable selection of the base material.

In an associated assignment table 22 (lookup or mapping table) or color table, an ideally unambiguous assignment between color and expiry period is defined with regard to the shelf life and the expiry date of the infusion tube 2, whereby certain groups or categories can be formed for simplification and to reduce the necessary distinguishable colors; in a very simple form, for example: red—shelf life until the end of 2025, green—shelf life until the end of 2027, blue—shelf life until the end of 2029.

Simultaneously or alternatively, in such an associated assignment table 22 (lookup or mapping table) or color table, an ideally unambiguous assignment between color and expiration time can be defined with regard to a dwell time of the infusion tube 2 in the sense of a maximum usage time as expiration time. Here, too, certain groups or categories can be formed to simplify and reduce the necessary distinguishable colors; in a very simple form, for example: pink—total usage time 96 hours, gray—total usage time 5 hours.

It is also within the scope of the present invention that, by means of such color coding, several parameter combinations are stored simultaneously in an associated assignment table 22 (lookup or mapping table) or color table so that they can be clearly assigned, for example:
Yellow—total service life 96 hours, shelf life until the end of 2025,
Orange—total service life 5 hours, shelf life until the end of 2025,
light blue—Total service life 96 hours, expires at the end of 2027,
Dark blue—total service life 96 hours, expires at the end of 2029

In practice, depending on the type of sensor used for color detection (see below), different colors can be distinguished according to their hue, saturation and/or brightness (or alternative characterizations).

The assignment table 22 is advantageously signed and/or encrypted and stored in the pump controller 24 of the infusion pump 16 or another evaluation unit communicating with the infusion pump 16 and is thus protected on the one hand against easy readout and on the other hand against easy manipulation by unauthorized persons.

The infusion pump 16, into which the infusion tube 2 with the sliding clamp 10 is inserted for use, is equipped with means for automated, mechanical detection and decoding/decoding of the code attached to or arranged on the sliding clamp 10. Using the above-mentioned assignment table 22, which is stored or saved in a memory unit of the pump controller 24 (or evaluation unit), an algorithm implemented in the pump controller 24 determines the shelf life or expiry date of the infusion tube 2 from the information contained in the code and compares it with the current system time, which has previously been compared at least once with the external time and is advantageously synchronized with it on a regular basis. In addition or alternatively, a dwell time of the disposable item can be recorded and, if necessary, elapsed usage time can be stored. If this comparison shows that the expiry time, in particular the dwell time, the shelf life or the expiry date, possibly including a predefined grace period, has been exceeded, the pump controller 24 can output a signal to the user, for example via a user interface, which is preferably suitable for blocking or allowing an infusion operation with the disposable article 4. Alternatively, the signal may not be output to the user interface or the user. In other words, for example, further operation can be permitted without the user being specifically informed of this. Additionally or alternatively, the signal may include further information for the user. For example, the signal can be a warning message to the user. The signal can also be a remaining expiry time, in particular a remaining dwell time or a period of time up to the shelf life or an expiry date, In particular, the signal can be a warning message or a request to change the tube and the pump controller 24 refuses or blocks or stops operation with the old tube. Otherwise, operation can continue as normal.

The described check can, for example, always be carried out after a change of the infusion tube 2 or after opening/reclosing the closure flap 18 to indicate such a change. During operation, on the other hand, it is advisable not to carry out the check so as not to jeopardize a running infusion by interrupting or blocking it. At the same time, the expiry time can also be monitored regularly or continuously during operation in the form of the maximum dwell time.

It can also be provided in the pump controller 24 that an authorized user, for example after entering an authorization code via a keypad or after other authorization, is able to remove a blockage, for example in order to carry out an infusion with an outdated infusion tube (beyond the expiration date, in particular the expiration date) in emergency situations (e.g. disaster or war with corresponding supply bottlenecks). It may also be possible to use an item beyond the maximum retention period.

To detect the color of the color code 26, an optical sensor, in particular a color sensor 26 with an associated illumination unit 28, is expediently integrated into the infusion pump 16. For example, the components can be arranged in such a way that light emitted by the illumination unit 28 falls on the sliding clamp 10 seated in the receptacle 30 and is reflected by the latter, with the reflected light falling on the color sensor 16. The color sensor then evaluates a reflection spectrum, for example. Alternatively, a transmission spectrum can be evaluated if the components are arranged accordingly, provided that the element carrying the color coding 26 is sufficiently translucent. In both cases (reflection or transmission), illumination by ambient light can in principle be used instead of illumination by an illumination unit 28, although this is not always practicable due to the fluctuating ambient light conditions and is at least more prone to errors.

Instead of and/or in addition to a conventional color in the visible range of the optical spectrum, a spectral property of an emission spectrum and/or transmission spectrum in the non-visible spectral range can also be used to identify the sliding clamp 10 and to encode the shelf life or the expiry date. Accordingly, the illumination unit 28 can be designed to emit IR or UV light, for example. The same applies to the spectral sensitivity of the color sensor 16, which is adapted to the wavelength of interest. The excitation and evaluation of fluorescence effects as a carrier of the coding is also conceivable.

The color sensor can be designed in such a way that it determines the color of the color coding and communicates it to the evaluation algorithm or the evaluation unit of the pump controller. However, it may also be the case that only certain physical properties of the reflected or transmitted light are measured by the color sensor, with the actual evaluation and determination of the color then taking place in the pump controller 28 or evaluation unit. Instead of an evaluation unit integrated into the infusion pump 16, an embodiment may also be provided in which the corresponding functionality is at least partially outsourced to an external component and is provided, for example, by a central computer or cloud service.

In a modification of the concept described, the color coding 26 used to determine the expiry time, in particular the dwell time, the shelf life or the expiry date, can be arranged at a location other than on/at a sliding clamp 10. For example, a marking flag or other marking connected or connectable to the infusion tube 2 or another corresponding accessory is possible. Furthermore, a color imprint or a color layer or a material-related color coding directly on the surface of the infusion tube 2 is a possible alternative.

Instead of a color code 26, character codes printed or otherwise attached or arranged on the infusion tube 2, the sliding clamp 10 or another accessory can also be used, which can be read/evaluated by the infusion pump 16 via an optical sensor and thus represent the function described above. In particular, to determine an expiry time in terms of a maximum retention time, it may be useful to make the disposable article uniquely identifiable by means of corresponding coding in order to ensure that the expiry time of the specific disposable article can be correctly recorded. In general, the term “character code” refers to any type of coded representation of information that is based on visual elements, whether through special symbols, graphic patterns or other visual markings. In particular, barcodes and QR codes can be considered as information carriers: these are types of character codes that use visual patterns to represent information. Barcodes consist of a series of parallel lines of different widths, while QR codes typically consist of a pattern of square blocks. Both are captured by sensor-based readers and converted into digital data.

Non-standardized or non-standard (in-house) coding can be used to make it more difficult for unauthorized persons to read and manipulate.

As a further alternative, RFID tags, which contain the expiry time, in particular the dwell time, the shelf life or the expiry date of the infusion tube 2 in coded form, can be integrated into the infusion tube 2, the sliding clamp 10 or the other accessory. The information stored on the RFID tag can be read out by a suitable sensor of the infusion pump 16 and evaluated and used as described above. The RFID tag can also contain a dedicated copy protection sequence. However, the RFID tag makes the infusion tube 2 more expensive to manufacture, as it has to be integrated into the infusion tube 2.

Instead of a sliding clamp 10, which regulates the flow of liquid through the infusion tube 2 by displacement relative to the infusion tube 2, any other tube clamp with an equivalent function can be used, such as a swivel or hinged clamp. In this case, too, the receptacle 30 and/or closure flap 18 of the infusion pump 16 are expediently designed in such a way that they bring the hose clamp into the open or closed position when it is inserted into and/or removed from the infusion pump 16.

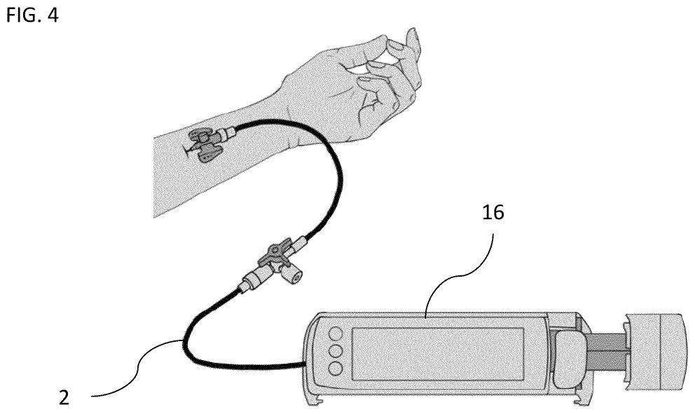

Instead of an infusion tube 2, an infusion bag or a syringe for connecting or inserting into/onto the infusion pump 16 can also be provided as a disposable article 4 and correspondingly equipped with a color code 20 or other coding. This means that all the concepts described above using the example of a peristaltic pump for the automatic detection and consideration of an expiry time, in particular a dwell time, a shelf life or an expiry date by means of a coded hose or slide clamp can be transferred analogously to a syringe pump, whereby in this case preferably a correspondingly coded medical syringe, which is inserted into the pump, causes the activation. This variant is illustrated in FIG. 4. It is preferred that the syringe plunger and/or the hollow syringe body bear the code and, in particular, are colored or marked in color.

Another name for a syringe pump is a piston pump. In contrast to the peristaltic pump, which works by squeezing and releasing a flexible tube, the syringe pump uses a piston mechanism (inside the syringe) to pump liquid. A piston moves back and forth in a cylinder to draw in and deliver liquid.

### LIST OF REFERENCE SYMBOLS

2 Infusion tube
4 Disposable article
6 Drip chamber or infusion needle end
8 Infusion bag or infusion pump end
10 Sliding clamp
12 Frame
14 Elongated hole
16 Infusion pump
18 Closure flap
20 Color code
22 Assignment table
24 Pump controller
26 Color sensor
28 Illumination unit
30 Receptacle
32 Infusion bag
34 Roller clamp

1. A medical infusion system comprising:
at least one disposable article;
at least one infusion pump having a receptacle or a connection for the at least one disposable article,
the at least one disposable article configured for insertion into or connection to the at least one infusion pump,
the at least one disposable article comprising or connected to a machine-readable code that contains or represents an expiry time of the at least one disposable article,
the at least one infusion pump comprising a sensor for detecting the machine-readable code,
the at least one infusion pump further comprising a pump controller that is configured or programmed to determine the expiry time of the at least one disposable article based on the machine-readable code.

2. The medical infusion system according to claim 1, wherein the at least one disposable article comprises an infusion tube.

3. The medical infusion system according to claim 2, wherein the infusion tube is connected to an accessory, and the accessory comprises the machine-readable code.

4. The medical infusion system according to claim 3, wherein the accessory is a sliding clamp.

5. The medical infusion system according to claim 4, wherein the at least one infusion pump has a closure flap that actuates the sliding clamp.

6. The medical infusion system according to claim 1, wherein the at least one disposable article comprises a syringe.

7. The medical infusion system according to claim 1, wherein the expiry time comprises a retention time of, a shelf life of, or an expiry date of the at least one disposable article.

8. The medical infusion system according to claim 1, wherein the pump controller is configured or programmed to:
use the machine-readable code to determine the expiry time of the at least one disposable article,
compare the expiry time with a system time to make a comparison, and
depending on the comparison, generate a signal to a user.

9. The medical infusion system according to claim 8, wherein the signal is operable to block or permit an infusion operation with the at least one disposable article.

10. The medical infusion system according to claim 9, wherein, when the infusion operation is blocked, the pump controller is configured to output the signal as a warning message via a user interface.

11. The medical infusion system according to claim 9, wherein, when the infusion operation is blocked, the pump controller is configured to permit an authorized user to resume the infusion operation by entering a code.

12. The medical infusion system according to claim 8, wherein the pump controller is configured to synchronize the system time with an external time.

13. The medical infusion system according to claim 1, wherein the machine-readable code is a color code.

14. The medical infusion system according to claim 13, wherein the sensor is a color sensor.

15. The medical infusion system according to claim 1, wherein the machine-readable code is a graphic code.

16. The medical infusion system according to claim 1, wherein the machine-readable code is carried by an RFID tag.

17. The medical infusion system according to claim 1, wherein an assignment table is stored in the medical infusion system, and wherein the assignment table links the machine-readable code with the expiry time.

18. The medical infusion system according to claim 17, wherein the assignment table is stored in the pump controller.

19. The medical infusion system according to claim 17, wherein the assignment table is digitally signed and/or stored in encrypted form.

20. A method for operating the medical infusion system according to claim 1, the method comprising the steps of:
inserting the at least one disposable article into, or connecting the at least one disposable article to, the at least one infusion pump;
detecting the machine-readable code that contains or represents the expiry time of the at least one disposable article;
comparing the expiry time with a system time; and
blocking operation of the at least one infusion pump when the system time exceeds the expiry time.
